(19) United States
(12) Patent Application Publication
(10) Pub. No.: US 2023/0309565 A1
(43) Pub. Date: Oct. 5, 2023

(54) PSEUDOMONAS STRAINS AND THEIR METABOLITES TO CONTROL CITRUS HUANGLONGBING AND RELATED DISEASES

(71) Applicants: T3 BIOSCIENCE, LLC, MEQUON, WI (US); UWM RESEARCH FOUNDATION, INC., MILWAUKEE, WI (US)

(72) Inventors: Ching-Hong Yang, Mequon, WI (US); Jian Huang, Milwaukee, WI (US)

(21) Appl. No.: 18/120,799

(22) Filed: Mar. 13, 2023

Related U.S. Application Data
(60) Provisional application No. 63/319,287, filed on Mar. 12, 2022.

(51) Int. Cl. A01N 63/27; A01H 6/78; A01H 1/00

(52) U.S. Cl. CPC A01N 63/27; A01H 1/125; A01H 6/785

(57) ABSTRACT

The present disclosure concerns methods of using novel bacterial strains of 0617-T307, 0917-T305, 0917-T306, 0917-T307, 0118-T319, 0318-T327, and 0418-T328, the cell broth and novel metabolites produced from the bacterial strains, to inhibit the growth of a variety of microbial species for bacterial pathogens that cause citrus Huanglongbing (citrus greening disease), Zebra chip (ZC) disease of potato, tomato, and other plants of the family Solanaceae and plants of the family Apiaceae and Umbelliferae. The methods include use of novel, potent antimicrobial metabolites produced from the strains corresponding to a Pseudomonas bacterial metabolite as Formula (I):

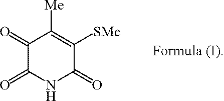 [SMILES: CSC1=C(C)C(=O)C(=O)NC1=O]

### CROSS-REFERENCE TO RELATED APPLICATIONS

This application claims benefit of priority to U.S. Provisional Pat. Application Serial No. 63/319,287, filed Mar. 12, 2022 and entitled “PSEUDOMONAS STRAINS AND THEIR METABOLITES TO CONTROL CITRUS HUANGLONGBING AND RELATED DISEASES,” the contents of which are herein incorporated by reference in its entirety.

### FIELD OF THE INVENTION

This invention is in the field of biopesticides. In particular, the invention pertains to seven novel strains of Pseudomonas spp., 0617-T307, 0917-T305, 0917-T306, 0917-T307, 0118-T319, 0318-T327, and 0418-T328, the cell broth and novel metabolites produced from the bacterial strain that can inhibit the growth of a variety of microbial species. The Pseudomonas strains of 0617-T307, 0917-T305, 0917-T306, 0917-T307, 0118-T319, 0318-T327, and 0418-T328 have been deposited in the American Type Culture Collection (ATCC) and have ATCC accession number PTA-126796, PTA-126797, PTA-126798, PTA-126799, PTA-126800, PTA-126801, and PTA-126802, respectively.

### BACKGROUND OF THE INVENTION

Plant diseases caused by pathogenic microorganisms are exponentially increasing and cost-consuming. The plant pathogenic organisms include fungus, bacterium, mycoplasma, virus, viroid, nematode, or parasitic flowering plant. Currently, there are 14 common plant diseases caused by bacterial organisms, including bacterial spot, bacterial blight, bacterial wilt, etc. Huanglongbing (HLB), also known as citrus greening, is caused by the bacteria Candidatus Liberibacter asiaticusis, Ca. Liberibacter americanus and Ca. Liberibacter africanus. HLB is the most serious disease of citrus that affects citrus production in Asia, Africa, the Indian subcontinent, and the Arabian Peninsula (Bové (2006). HLB is thought to have originated in Asia and was first detected in the United States occurred in Florida in 2005. Since 2005, HLB has spread through the citrus-producing areas in Florida, reducing citrus production by 75%, while more than doubling the cost of production. In 2008, HLB was detected in Louisiana, and in 2009, the disease was detected in Georgia and South Carolina. In 2012, HLB was detected in Texas and residential areas of California (Hu & Wright. (2019)). In Florida alone, the bacterial disease citrus greening has cost Florida’s economy an estimated $3.63 billion in lost revenues since 2006.

Microbial natural products have provided rich amounts of biological compounds as pesticides (Gwinn (2018)). However, current prevention methods for the HLB have limited effectiveness. The only products currently registered by EPA for use against citrus greening are FireWall, FireLine, and Mycoshield (oxytetracycline). The use of these products has declined due to a lack of confidence in their performance by growers. Also, concerns about antibiotic use, especially in fresh market citrus, have limited the use of these products.

In the last few decades, numerous non-antibiotic products have been developed, registered with the Environmental Protection Agency (EPA), National Organic Program (NOP)-approved, and marketed to orchardists for crop disease control (Tianna et al. (2018)). Prospective biological protection products must, on the one hand, effectively compete with the pathogens and, on the other, must be able to colonize the same niches on different organs of target plants. Protective bacteria produce secondary metabolites that affect the pathogens and compete for food and space, preventing pathogenesis by the plant pathogens in relation to the plant.

The metabolites produced by Gram-negative Pseudomonas species have been comprehensively reviewed (Masschelein et al. (2017)). The types of Pseudomonas metabolites can be classified as phenolic compounds, phenazine, lipopeptides, etc. The function of Pseudomonas species and their metabolites include the following (Alsohim et al. (2014)): 1) Produce hormones or induce systemic resistance; 2) Many naturally occurring strains also significantly improve plant growth (Plant growth regulator, IAA, viscosin); 3) Antagonism can be conferred by the production of siderophores and of surfactants, such as viscosin and viscosinamide, as well as antimicrobial compounds, such as hydrogen cyanide, phenazines, pyrrolnitrin or 2,4-diacetylphloroglucinol (DAPG). In our work, the bacterial strains were identified, the fermentates and novel metabolites were produced from the bacteria; specifically, RejuAgro A shows high potency on bacterial pathogens that cause HLB on citrus.

There is a need for new biopesticides derived from novel strains, cell broths, and novel metabolites produced from such strains that can inhibit the growth of various crop pathogens.

### BRIEF SUMMARY OF THE INVENTION

In a first aspect, a method of controlling a crop disease is provided. The method includes several steps. A first step includes producing an agricultural composition that includes a Pseudomonas bacterial metabolite as Formula (I):

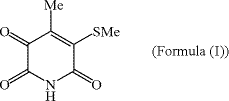 [SMILES: CSC1=C(C)C(=O)C(=O)NC1=O]

A second step includes applying the agricultural composition to a crop to inhibit the growth of a pathogenic microorganism.

In a second aspect, a method of controlling a crop disease is provided. The method includes a step of applying an agriculture composition that includes between about 1.0 × 10⁵ and 1.0 × 10⁹ cfu per mL Pseudomonas bacteria to a crop to inhibit the growth of a pathogenic microorganism.

In a third aspect, a method to activate the expression of a defense marker gene in a citrus crop is provided. The method includes several steps. A first step i producing an agricultural composition comprising a Pseudomonas bacterial metabolite as Formula (I)

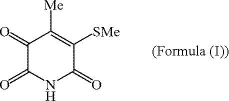 [SMILES: CSC1=C(C)C(=O)C(=O)NC1=O]

A second step includes applying the agricultural composition to the citrus crop, wherein the result is to activate expression of the defense marker gene.

In a fourth aspect, a method to activate the expression of a defense marker gene in a citrus crop is provided. A first step includes applying an agricultural composition comprising between about 1.0 × 10⁵ and 1.0 × 10⁹ cfu per mL Pseudomonas bacteria to the citrus crop, wherein the result is to activate the expression of the defense marker gene in the citrus crop.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIG. 1 illustrates an example of assay-guided isolation of ethyl acetate extract of strain 0617-T307.

FIG. 2A depicts exemplary culture plots showing the amount of RejuAgro A in a shaking flask fermentation in which the distribution of RejuAgro A in the cell broth, supernatant, and cells.

FIG. 2B depicts an exemplary plot of the production of RejuAgro A from cell fermentation over time.

FIG. 3 depicts an exemplary amount-peak area curve of RejuAgro A analyzed by HPLC at the wavelength of 406 nm.

FIG. 4 depicts exemplary data on RejuAgro A production from different bacterial strains.

FIG. 5A depicts inhibition of growth of L. crescens BT1 in BM7 medium at 28° C. following application of a dosage of RejuAgro A (RAA: 10 mg/L, 20 mg/L, and 40 mg/L)

FIG. 5B depicts partial inhibition of growth of L. crescens following application of RejuAgro A (RAA) at dosage of 1 mg/L.

FIG. 5C depicts inhibition of growth of L. crescens for 16 days following application of RejuAgro A (RAA) at a dosage of 2.5 mg/L, which is comparable to oxytetracycline (Oxy-Tet) and streptomycin (Str) at the same concentration.

FIG. 6 depicts the RejuAgro A (RAA) at 10 mg/L can effectively suppress the Candidatus Liberibacter asiaticus (CLas) when sprayed on the leaves of citrus Huanglongbing positive trees.

FIG. 7A depicts qRT-PCR results showing expression of CsPR1 in the midribs of citrus leaves after the RejuAgro treatment at 0 (CK), 10 ppm (R10), and 20 ppm (R20) concentration. Values were normalized as relative expressions to CsUbi. The statistical significance was evaluated by two-sided Student’s t test; Error bars: SD; *p < 0.05, **p < 0.01, and ***p < 0.001.

FIG. 7B depicts qRT-PCR results showing expression of CsPR2 in the midribs of citrus leaves after the RejuAgro treatment at 0 (CK), 10 ppm (R10), and 20 ppm (R20) concentration. Values were normalized as relative expressions to CsUbi. The statistical significance was evaluated by two-sided Student’s t test; Error bars: SD; *p < 0.05, **p < 0.01, and ***p < 0.001.

### DETAILED DESCRIPTION

The present invention relates to a novel metabolite produced by seven Pseudomonas strains listed in this patent, such as 0617-T307, that exhibits antimicrobial activity against pathogenic microorganisms. From the 16S rRNA and other housekeeping gene sequences, the strain was identified as Pseudomonas soli 0617-T307 in the Pseudomonas putida group. The cell broth of the 7 bacterial strains, such as 0617-T307, contains a novel, potent 6-membered heterocycle natural product which is designated as RejuAgro A, as depicted below:

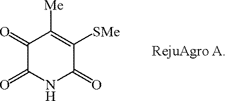 [SMILES: CSC1=C(C)C(=O)C(=O)NC1=O]

This compound, their method of production, and applications for inhibiting plant microbial pathogens are disclosed in greater detail herein.

### Definitions

When introducing elements of aspects of the disclosure or particular embodiments, the articles “a,” “an,” “the,” and “said” are intended to mean that there are one or more of the elements. The terms “comprising,” “including,” and “having” are intended to be inclusive and mean that there may be additional elements other than the listed elements. The term “or” means any one member of a particular list and also includes any combination of members of that list, unless otherwise specified.

As intended herein, the terms “substantially,” “approximately,” and “about” and similar terms are intended to have a broad meaning in harmony with the common and accepted usage in the art to which the subject matter of this disclosure pertains. It should be understood by those of skill in the art who review this disclosure that these terms are intended to allow a description of certain features described and claimed without restricting the scope of these features to precise numerical ranges provided. Accordingly, these terms should be interpreted as indicating that insubstantial or inconsequential modifications or alterations of the subject matter described and claimed are considered to be within the scope of the invention as recited in the appended claims.

“Biological control agents (or BCAs)” are a way of managing pests, such as pathogens, weeds and insects, safely, sustainably, and cost-effectively. These agents are introduced into the environment to target a pest species, with the aim of reducing the pest’s population or abundance in the environment.

“Biologicals” are preparations of living microorganisms (e.g., bacteria and fungi) that produce colonies on the hosts. These microorganisms are applied mainly to slow the pathogen buildup during the epiphytic phase (Tianna et al. (2018)).

“Biorational” is a term applied to microbe-based biopesticides. These biopesticides are often made by fermenting microbial strains. Most of these products have both anti-bacterial and anti-fungal activity (Tianna et al. (2018)).

“Biopesticides” is defined by The US Environmental Protection Agency (EPA) to be pesticides derived from natural materials and categorizes them as either biochemical pesticides, containing substances that control pests by nontoxic mechanisms, microbial pesticides, consisting of microorganisms that typically produce bioactive natural products (BNPs), or plant-incorporated-protectants with activity produced by plants because of added genetic materials (Gwinn (2018)).

The compound referred to as RejuAgro A, corresponds to chemical compound having Formula (I), as illustrated below:

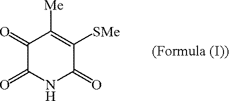 [SMILES: CSC1=C(C)C(=O)C(=O)NC1=O]

In a first aspect, a method of controlling a crop disease is provided. The method includes several steps. A first step includes producing an agricultural composition comprising a Pseudomonas bacterial metabolite as Formula (I):

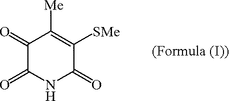 [SMILES: CSC1=C(C)C(=O)C(=O)NC1=O]

A second step includes applying the agricultural composition to a crop to inhibit the growth of a pathogenic microorganism.

In one respect, the method includes a crop disease selected from the group consisting of citrus Huanglongbing disease, Zebra chip (ZC) disease of potatoes, tomatoes, and other plants of the family Solanaceae and plants of the family Apiaceae and Umbelliferae. In a second respect, the method includes the pathogenic microorganism selected from the group consisting of Liberibacter crescens, Candidatus Liberibacter asiaticus, Ca. Liberibacter americanus, Ca. Liberibacter africanus, and Ca. Liberibacter solanacearum. In a third respect, the method includes the crop selected from the group consisting of all citrus species and hybrids, as well as numerous citrus relatives in the family Rutaceae, Solanaceae, potatoes, tomatoes, and other plants of the family Solanaceae and plants of the family Apiaceae and Umbelliferae.

In a second aspect, a method of controlling a crop disease is provided. The method includes a step of applying an agricultural composition comprising between about 1.0 × 10⁵ and 1.0 × 10⁹ cfu per mL Pseudomonas bacteria to a crop to inhibit the growth of a pathogenic microorganism.

In a first respect, the method includes the Pseudomonas bacteria selected from the group consisting of Pseudomonas soli 0617-T307 (Accession No. PTA-126796), Pseudomonas soli 0917-T305 (Accession No. PTA-126797), Pseudomonas soli 0917-T306 (Accession No. PTA-126798), Pseudomonas soli 0917-T307 (Accession No. PTA-126799), Pseudomonas mosselii 0118-T319 (Accession No. PTA-126800), Pseudomonas mosselii 0318-T327 (Accession No. PTA-126801), and Pseudomonas mosselii 0418-T328 (Accession No. PTA-126802). In a second respect, the method includes an agricultural composition of between about 5.0 × 10⁷ and 2.0 × 10⁸ cfu per mL Pseudomonas bacteria. In a third respect, the method includes the crop disease selected from the group consisting of citrus Huanglongbing disease, Zebra chip (ZC) disease of potatoes, tomatoes, and other plants of the family Solanaceae and plants of the family Apiaceae and Umbelliferae. In a fourth respect, the method includes the pathogenic microorganism selected from the group consisting of Liberibacter crescens, Candidatus Liberibacter asiaticus, Ca. Liberibacter americanus, Ca. Liberibacter africanus, and Ca. Liberibacter solanacearum. In a fifth respect, the method includes the crop being from the group consisting of all citrus species and hybrids, as well as numerous citrus relatives in the family Rutaceae, Solanaceae and other plants of the family Solanaceae, potatoes, tomatoes, and plants of the family Apiaceae and Umbelliferae.

In a third aspect, a method to activate the expression of a defense marker gene in a citrus crop is provided. The method includes several steps. A first step i producing an agricultural composition comprising a Pseudomonas bacterial metabolite as Formula (I)

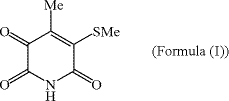 [SMILES: CSC1=C(C)C(=O)C(=O)NC1=O]

A second step includes applying the agricultural composition to the citrus crop, wherein the result is to activate expression of the defense marker gene.

In a first respect, the citrus crop is selected from the group consisting of a citrus species, a hybrid derived from a citrus species, a citrus plant species relative in the family Rutaceae, and a citrus plant species relative of the family Solanaceae. In a second respect, the defense marker gene is selected from CsPR1 and CsPR2.

In a fourth aspect, a method to activate the expression of a defense marker gene in a citrus crop is provided. A first step includes applying an agricultural composition comprising between about 1.0 × 10⁵ and 1.0 × 10⁹ cfu per mL Pseudomonas bacteria to the citrus crop, wherein the result is to activate the expression of the defense marker gene in the citrus crop.

If a first respect, the Pseudomonas bacteria is selected from the group consisting of Pseudomonas soli 0617-T307 (Accession No. PTA-126796), Pseudomonas soli 0917-T305 (Accession No. PTA-126797), Pseudomonas soli 0917-T306 (Accession No. PTA-126798), Pseudomonas soli 0917-T307 (Accession No. PTA-126799), Pseudomonas mosselii 0118-T319 (Accession No. PTA-126800), Pseudomonas mosselii 0318-T327 (Accession No. PTA-126801), and Pseudomonas mosselii 0418-T328 (Accession No. PTA-126802). In a second respect, the agricultural composition includes between about 5.0 × 10⁷ and 2.0 × 10⁸ cfu per mL Pseudomonas bacteria. In a third respect, the citrus crop is selected from the group consisting of a citrus species, a hybrid derived from a citrus species, a citrus plant species relative in the family Rutaceae, and a citrus plant species relative of the family Solanaceae. In a fourth respect, the defense marker gene is selected from CsPR1 and CsPR2.

### Biological Deposit Information

One of the inventors, Dr. Ching-Hong Yang (residing at 10120 N. Sheridan Dr., Mequon, WI 53902, US), submitted bacterial strains Pseudomonas soli 0617-T307, Pseudomonas soli 0917-T305, Pseudomonas soli 0917-T306, Pseudomonas soli 0917-T307, Pseudomonas mosselii 0118-T319, Pseudomonas mosselii 0318-T327, and Pseudomonas mosselii 0418-T328 to the American Type Culture Collection (ATCC®), P.O. Box 1549, Manassas, VA 20110 USA (“ATCC Patent Depository”) on Jun. 25, 2020, as evidenced by Form PCT/RO/134, “Indications Relating to Deposited Microorganism,” pursuant to PCT Rule 13bis (filed in this application). Following viability testing, the ATCC Patent Depository accorded these deposited bacterial strains the following Accession numbers, effective Jun. 25, 2020: Pseudomonas soli 0617-T307 (Accession No. PTA-126796), Pseudomonas soli 0917-T305 (Accession No. PTA-126797), Pseudomonas soli 0917-T306 (Accession No. PTA-126798), Pseudomonas soli 0917-T307 (Accession No. PTA-126799), Pseudomonas mosselii 0118-T319 (Accession No. PTA-126800), Pseudomonas mosselii 0318-T327 (Accession No. PTA-126801), and Pseudomonas mosselii 0418-T328 (Accession No. PTA-126802). Dr. Yang grants permission to Applicants to include this biological deposit disclosure in the present application and gives his unreserved and irrevocable consent to it being made available to the public as of the date of filing.

### EXAMPLES

### Example 1. Preparation, Isolation, and Characterization of RejuAgro A From Ethyl Acetate Extracts of the Cell Broth of Strain 0617-T307.

The preparation of RejuAgro A (Formula (I)) can be obtained by ethyl acetate extraction of the cell broth from the fermenter fermentation, followed by the chromatographic isolation and purification. Briefly, the stock bacterium Pseudomonas sp. 0617-T307 was streaked onto LB plate (Tryptone, 10 g/L; Yeast extract, 5 g/L; NaCl, 10 g/L; agar, 15 g/L; water) and grew in a 28° C. incubator for 24 h. For the preparation of seed media, single colony of 0617-T307 was inoculated into a 2.0 L flask containing 500 mL autoclaved YME media (yeast extract, 4 g/L; glucose 4 g/L and malt extract 10 g/L) and grow at 28° C. for 24 h in a shaking speed of 200 rpm. Then the seed media was inoculated into a 20 L NBS fermenter containing 12 L autoclaved YME media. The fermentation was proceeded at 16° C. for 1-7 days. The agitation speed and the airflow rate were 200 rpm and 2 L/min, respectively.

After harvesting, the bacterial culture was extracted by ethyl acetate for four times. The ethyl acetate layer was separated and dehydrated using sodium sulfate and dried by rotary evaporation at 35° C. This resulted in 2.9 g crude extract from 12 L culture of strain 0617-T307.

The concentrated sample was dissolved in ethyl acetate and mixed with silica gel, which was packed as an injection column (φ3.0 × 20 cm) and mounted atop a silica gel Universal Column (4.8 × 18.5 cm) on a flash chromatography system (Yamazen AI-580) equipped with an UV detector. After loading the sample, the sample was eluted by the 280 mL of each of the following solvents in order with an increasing polarity, 100% hexane, 75% hexane/25% ethyl acetate, 50% hexane/50% ethyl acetate, 25% hexane/75% ethyl acetate, 100% ethyl acetate, 50% ethyl acetate/50% acetone, 100% acetone, and 100% methanol. The sample was eluted at a flow rate of 20 mL/min. The elute was monitored at UV 254 nm, and fractions were collected by a time mode at 20 mL/tubes. Totally, there are 114 fractions or tubes generated from the flash chromatography.

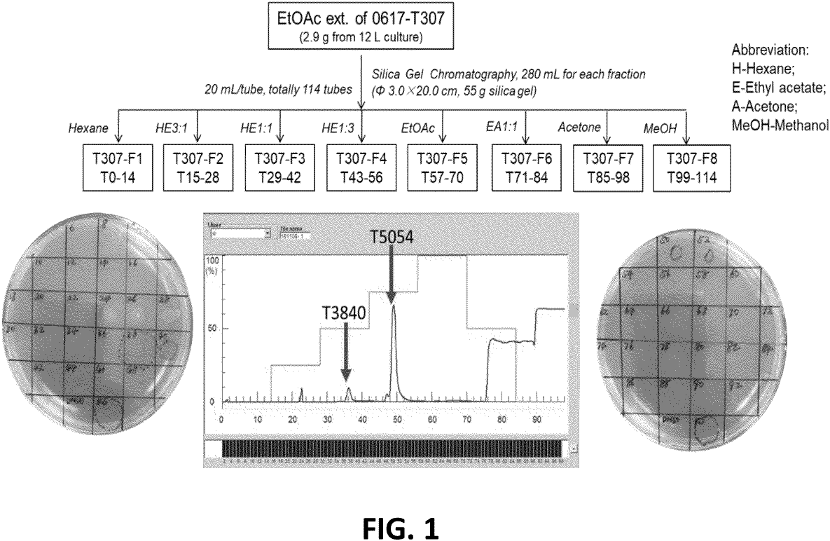

Fractions/tubes 38-40 (which was abbreviated as T3840 or Flash-RejuAgro A), which were eluded by 50% hexane/50% ethyl acetate (FIG. 1).

Preparative HPLC (Prep-HPLC) purification of the fraction 3840 and 5054 led to the discovery of 15 mg yellow-colored compound RejuAgro A (Rt17.5). RejuAgro A can be dissolved in methanol and chloroform. The structures of the compound have been investigated by High-resolution mass spectrometry (HR-MS), infrared (IR), Ultraviolet (UV), 1D and 2D Nuclear magnetic resonance (NMR) as well as X-ray crystal structure analysis. It showed that these two compounds are structurally similar, the compound RejuAgro A (Formula (I)) contain 7 types of carbon groups (three types carbonyl, two types tertiary carbons, two types of methyl carbons) as shown below:

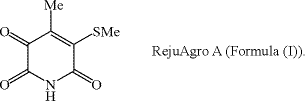 [SMILES: CSC1=C(C)C(=O)C(=O)NC1=O]

### Example 2. Production and Stability of RejuAgro A From Strain 0617-T307 in a Shaking-Flask Fermentation

The fermentation of 0617-T307 for the production and preparation of RejuAgro A can be obtained by two approaches, the shaking-flask fermentation and fermenter fermentation. The fermenter fermentation was described in Example 1. In this example, the flask fermentation can be obtained as below. The stock bacterium Pseudomonas sp. 0617-T307 was streaked onto YME agar plate (yeast extract, 4 g/L; glucose 4 g/L and malt extract 10 g/L; agar, 15 g/L) and grew at 28° C. incubator for 24 h. The seed media were made by growing single colony of 0617-T307 in a 250 mL flask containing 50 mL sterile YME liquid media at 16° C. and 220 rpm for 24 h. Then the seed media were inoculated into 4 L flask containing 0.5 L sterile YME media at 4% ratio (v/v). Following the inoculation (2%, v/v) into eight 4-L flasks each containing 2 L YME media, the bacteria were grown at 16° C. in a shaker at 200-220 rpm for 1-7 days.

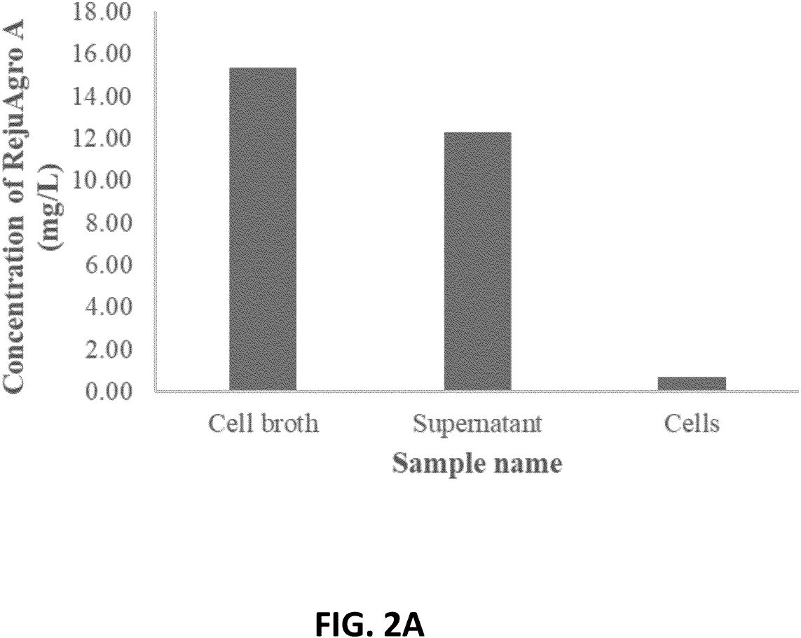

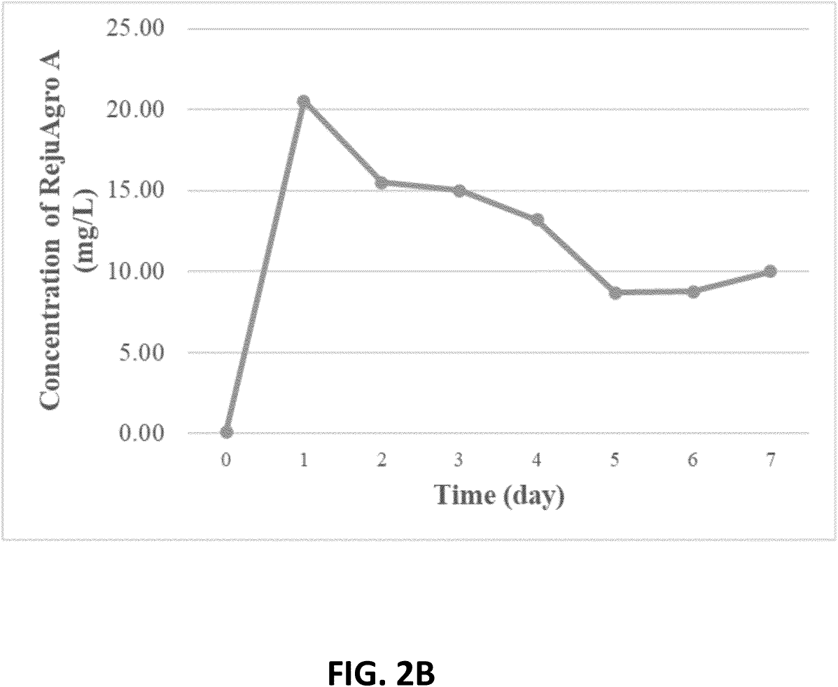

The RejuAgro A concentration was obtained by LC-MS analysis according to the developed standard curves. Two methods were used for the preparation of samples for LC-MS analysis. One approach is to extract the cell broth by ethyl acetate (1 mL:1 mL, vortex for 1 min), and to obtain the ethyl acetate extracts by centrifugation and vacuum drying of the ethyl acetate layer. The dried ethyl acetate extracts were dissolved in 40 µL methanol and 2 µL methanol solution was used for LC-MS analysis. The other method is to obtain the supernatant by centrifuging the cell broth, then mix the supernatant with equal volume of methanol to make the 50% methanol solution, of which 10 µL solution was injected into LC-MS. The second method was used because RejuAgro A production is an extracellular secretion process, which was demonstrated by the observation of the major amount of RejuAgro A in the supernatants rather than inside of the cells (FIG. 2A).

During the 7-day fermentation, the total production of RejuAgro A reached peak concentration on day one, then started to decrease with time increasing (FIG. 2B). Further detailed study on the production of RejuAgro A and the cell concentration were performed each 6 hours in shaking-flask fermentation. It showed that the concentration of RejuAgro A (total amount of RejuAgro A) reached to the maximum value of 13.8 mg/L at 18 h, and the concentration of bacteria cells reached to the maximum value of 2×10¹¹ CFU/mL at 12 h, which indicates the production of RejuAgro A is a cell growth-associated production process.

The volumes of the media in the 4-L shake-flasks affect the production of RejuAgro A. In the 4-L flasks with YME media, the production of RejuAgro A was only observed for the 500 mL volume size, and not observed for the 1.0 L or 1.5 L volume size. This observation indicates that the production of RejuAgro A prefers to occur in a highly aerated condition.

The media types and culture temperatures that affect the production of RejuAgro A. LB media was tested in parallel with YME media at 16° C. or 28° C. The production of RejuAgro A was observed in YME media but not in LB media at 16° C. Regarding the colony-forming-units, strain 0617-T307 grows well in LB media at both 16° C. and 28° C., and in YME media at 28° C. These results suggest that the production of RejuAgro A is both medium-specific and temperature-dependent. The activity for the products from 0617-T307 was monitored by plate assay against E. amylovora, which is consistent to the production of RejuAgro A.

To check the applicability of the production conditions for RejuAgro A, ten other Pseudomonas strains were tested under the same condition in parallel with the Pseudomonas strain 0617-T307. According to the analysis of housekeeping genes, 0917-T305, 0917-T306, and 0917-T307 were identified as Pseudomonas soli, and 0118-T319, 0318-T327, and 0418-T328 were identified as Pseudomonas mosselii. The type strains of both Pseudomonas. soli and Pseudomonas mosselii have been reported (Daboussi et al. (2002); Pascual et al. (2014)).

It showed that strain 0617-T307 and its phylogenetically closely related species can produce RejuAgro A in YME at 28° C. and 220 rpm. This result suggests that the method is specific for the strain 0617-T307 and some of its closely related species to produce RejuAgro A (Table 1). RejuAgro A can be present and stable in the culture at room temperature for at least 4 weeks, as tested by LCMS for 40-h culture obtained by growing 0617-T307 in YME media on a shaker at 16° C. and 220 rpm.

TABLE 1

Summary of RejuAgro A producing capabilities for the selected Pseudomonas strains that were cultured in medium YME at 16° C., 18 hours, 220 rpm

| | | |
|---|---|---|
| Strain code | Top-hit taxon | Production of RejuAgro A |
| 0617-T307 | Pseudomonas soli | Yes |
| 0617-T318 | Pseudomonas protegens | No |
| 0817-T317 | Pseudomonas protegens | No |
| 0717-T327 | Pseudomonas koreensis | No |
| 0717-T314 | Pseudomonas koreensis | No |
| 0917-T305 | Pseudomonas soli | Yes |
| 0917-T306 | Pseudomonas soli | Yes |
| 0917-T307 | Pseudomonas soli | Yes |
| 0118-T319 | Pseudomonas mosselii | Yes |
| 0318-T327 | Pseudomonas mosselii | Yes |
| 0418-T328 | Pseudomonas mosselii | Yes |

### Example 3. Identification and Characterization of the Bioactive Metabolites From Ethyl Acetate Extracts of the Supernatant of Strain 0617-T307.

The stock bacterium Pseudomonas sp. 0617-T307 was inoculated onto LB agar (Tryptone, 10 g/L; Yeast extract, 5 g/L; NaCl, 10 g/L; agar, 15 g/L; water) plate and grew at 28° C. incubator for 24 h. For the preparation of seed media, single colony of 0617-T307 was inoculated into 500 mL autoclaved YME media (yeast extract, 4 g/L; glucose 4 g/L and malt extract 10 g/L) and grow at 28° C. for 24 h in a shaking speed of 150 rpm. Then the seed media was inoculated into eight 4-L flasks each containing 2 L autoclaved YME media. The fermentation was proceeded at 16° C. in a shaker with a shaking speed of 150 rpm for 7 days. After 7-day growth, the supernatants were obtained by centrifuging bacterial culture at 4000 rpm for 15 min. The supernatants were then subjected to the ethyl acetate extraction. This resulted 3.0 g crude extract from 14 L culture of strain 0617-T307.

The concentrated sample was dissolved in acetone and mixed with silica gel, which was loaded to a silica gel column (φ3.0 × 20 cm) on a flash chromatography system (Yamazen AI-580) equipped with an UV detector. After loading the sample, the sample was eluted by the 280 mL of each of the following solvents in order with an increasing polarity, 100% hexane, 75% hexane/25% ethyl acetate, 50% hexane/50% ethyl acetate, 25% hexane/75% ethyl acetate, 100% ethyl acetate, 50% ethyl acetate 50% acetone, 100% acetone, and 100% methanol. The sample was eluted at a flow rate of 20 mL/min. The elute was monitored at UV 254 nm, and fractions were collected by a time mode at 20 mL/tubes. Totally, there are 114 fractions or tubes generated from the flash chromatography.

### Example 4 Production of RejuAgro A by Pseudomonas Species

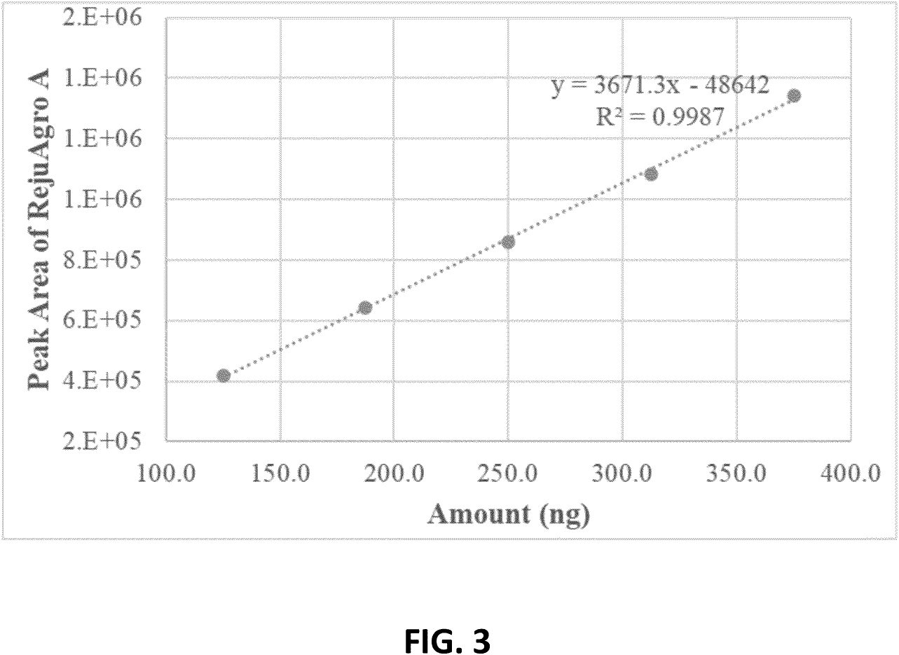

The amounts of RejuAgro A were analyzed by HPLC-MS for the broth after 24 h. fermentation in 4 L flask containing 500 mL YME media at 16° C. and 220 rpm shaking. The amount-peak area curve was prepared for-investigation of the relationship between HPLC peak area and the amount of RejuAgro A (FIG. 3). Analytical method: 1) 25 mL cell broth was extracted with 25 mL ethyl acetate; 2) 5 mL ethyl acetate extract was dried and dissolved in 0.1 mL methanol; 3) 4 µL was injected into HPLC-MS.

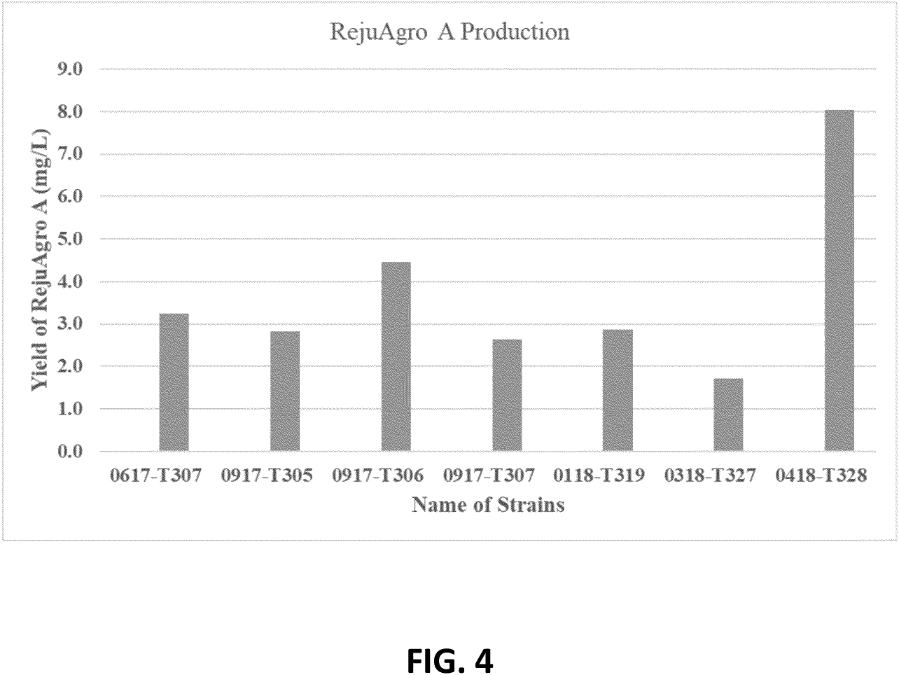

Seven bacteria (0617-T307, 0917-T305, 0917-T306, 0917-T307, 0118-T319, 0318-T327, 0418-T328) were evaluated for the production of RejuAgro A, the seed medium was prepared by growing the bacteria in YME medium at 16° C., 220 rpm for 24 h. HPLC analysis showed that all the seven bacteria produce RejuAgro A (FIG. 4).

### Example 5. Use of RejuAgro A for Inhibiting the Citrus Greening Disease, Zebra Chip Disease of Potatoes, and Other Solanaceous Hosts

Huanglongbing (HLB), also known as citrus greening, is the most devastating disease of citrus. The disease is caused by the bacterial pathogens Candidatus Liberibacter asiaticusis, Ca. Liberibacter americanus and Ca. Liberibacter africanus which are non-culturable in a pure medium. Liberibacter crescens is the only species of this genus that can be grown in axenic media, and it has been used as a model to study other non-culturable liberibacteral pathogens such as citrus greening causing Candidatus Liberibacter asiaticusis, Ca. Liberibacter americanus and Ca. Liberibacter africanus; and Ca. Liberibacter solanacearum, which causes Zebra chip (ZC) disease of potatoes and attacks tomatoes and other plants of the family Solanaceae and plants of the family Apiaceae or Umbelliferae (Sena-Vélez et al. (2019)).

HLB pathogens live in the phloem vessels of plants, and the spread of the disease requires insect vector Asian citrus psyllid. Efforts have been made to control the disease, but the effectiveness is limited and non-sustainable. Current methods to prevent infections and maintain the productivity of HLB-infected trees include insecticidal control of the vector, antibacterial treatments, and nutrient supplements. Antibiotics oxytetracycline and streptomycin are the only choices to show some efficacy in controlling the disease; however, these antibiotics could lead to antibiotic-resistant of human pathogens and disruption of the ecosystem of citrus trees. Insect control by spraying insecticides is also a potential threat to human health and non-targeted insects such as pollinating insects. The recent advance of using an antimicrobial peptide to treat HLB is promising (Huang et al. (2021)), but it is still in the experimental stage, and the costs of large-scale application into the vascular tissue of citrus trees could be high.

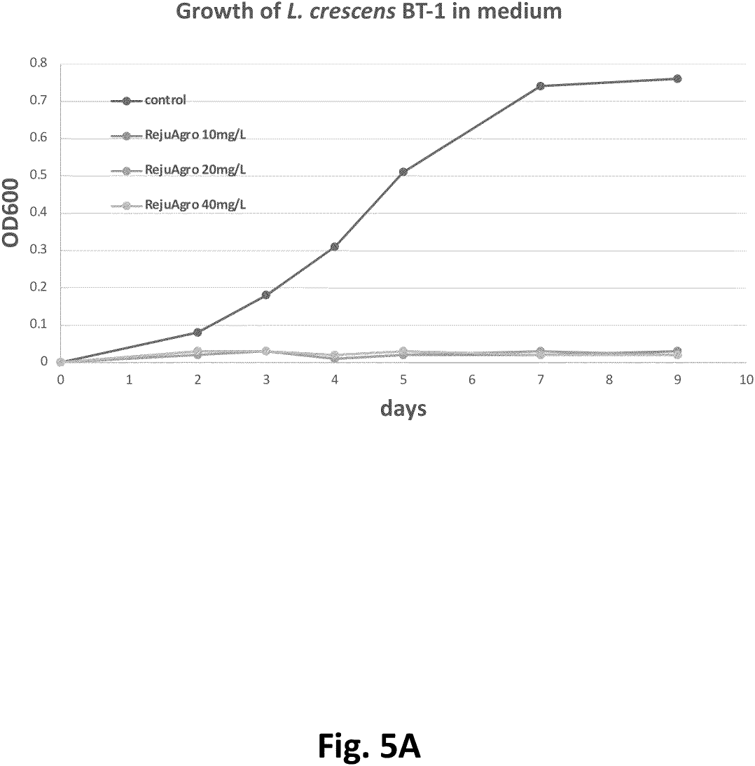

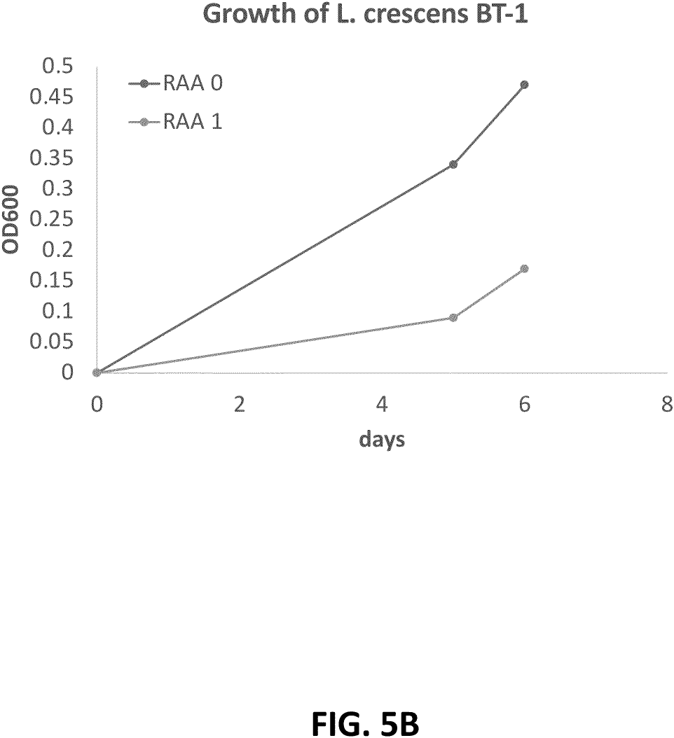

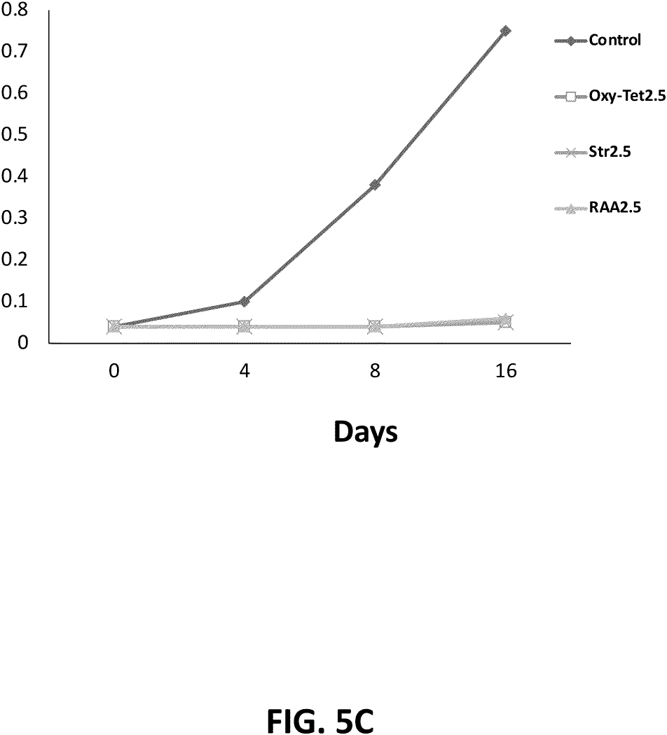

We have previously isolated a bacterial species Pseudomonas soli 0617-T307 from a soil sample in Wisconsin. The bacterial strain produces an active antimicrobial compound, namely RejuAgro A (RAA). We have successfully purified the RAA with the molecular weight of 185.2 g, which is notably smaller than oxytetracycline (MW: 460.4 g) and streptomycin (MW: 581.6 g). Results show that RAA can successfully inhibit the growth of Liberibacter crescens BT-1. The potency and minimal inhibitory concentration (MIC) is comparable to oxytetracycline and streptomycin which is as low as 2.5 mg/L for sixteen-day inhibition when grown in the BM7 medium (FIGS. 5A, B, and C). Liberibacter crescens is the only species of this genus that can be grown in axenic media, and it has been used as a model to study other non-culturable liberibacteral pathogens such as citrus greening causing Candidatus Liberibacter asiaticusis, Ca. Liberibacter americanus and Ca. Liberibacter africanus; and Ca. Liberibacter solanacearum, which causes Zebra chip (ZC) disease of potatoes and attacks tomatoes and other plants of the family Solanaceae and plants of the family Apiaceae or Umbelliferae (Sena-Vélez et al. (2019)). This result demonstrates RAA is highly potent to Liberibacter crescens, Candidatus Liberibacter asiaticusis, Ca. Liberibacter americanus and Ca. Liberibacter africanus; and Ca. Liberibacter solanacearum.

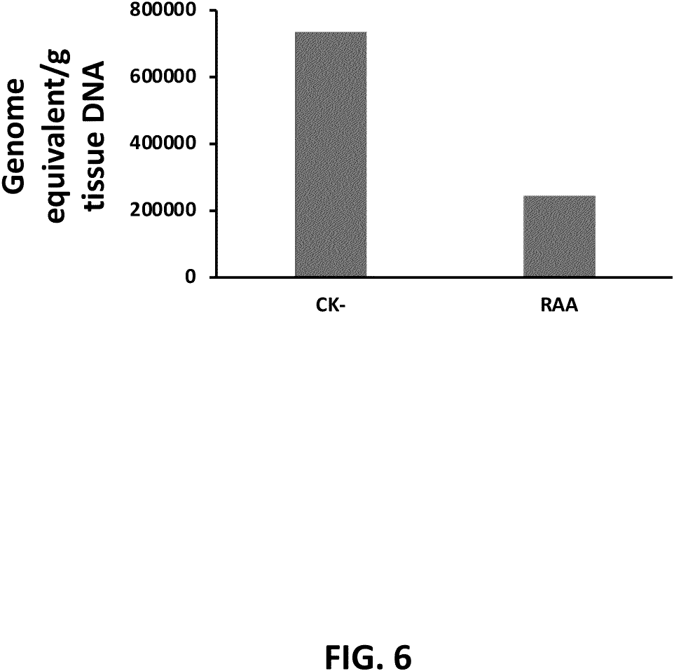

In the greenhouse assay, the RAA at 10 mg/L can effectively suppress the Candidatus Liberibacter asiaticus (“CLas”) when sprayed on the leaves of citrus Huanglongbing positive trees. The CLas titer in citrus leaves was calculated as reported (Ma et al. (2022)). Compared to the untreated control (CLas titer 7.4×10⁵ genome equivalent/g tissue DNA), we observed a rapid decrease in CLas titer (2.4×10⁵ genome equivalent/g tissue DNA) when we applied RAA by foliar spray onto the HLB-positive Duncan grapefruit (FIG. 6). The target of RAA will be plant bacterial diseases causing an economic loss in crops and fruits. As RAA is a novel and natural compound that has not been applied in human and animals, the risk of enhancing antibiotic-resistant would be much lower than other traditional antibiotics. In addition, the smaller molecular size will make it easier to reach the vascular tissues of citrus trees where HLB lives. RAA can be used to control HLB, Zebra chip, and diseases on many other solanaceous hosts caused by Candidatus with a significantly lower environmental impact.

### Example 6. RejuAgro Treatment in Citrus Plants Up-Regulates the Expression of Defense Marker Genes

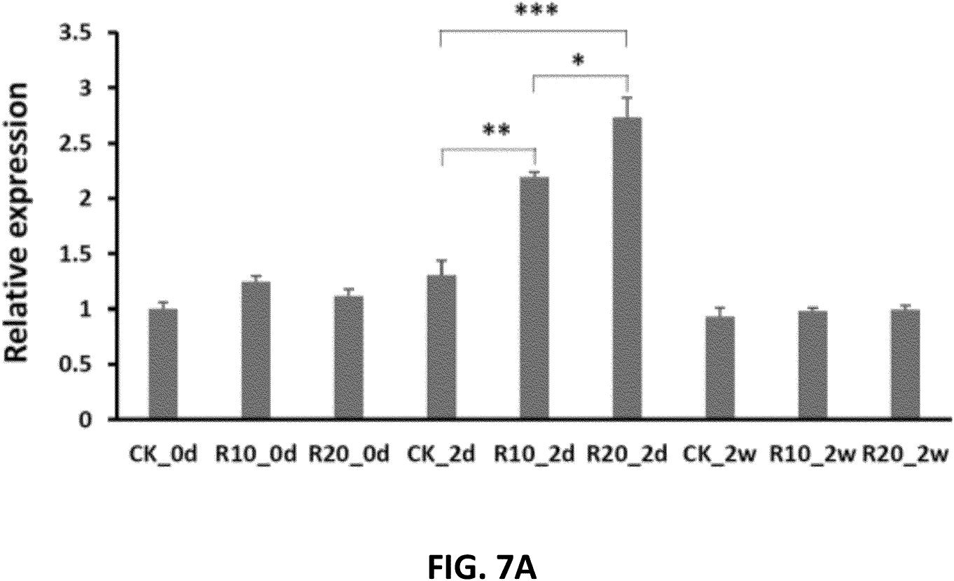

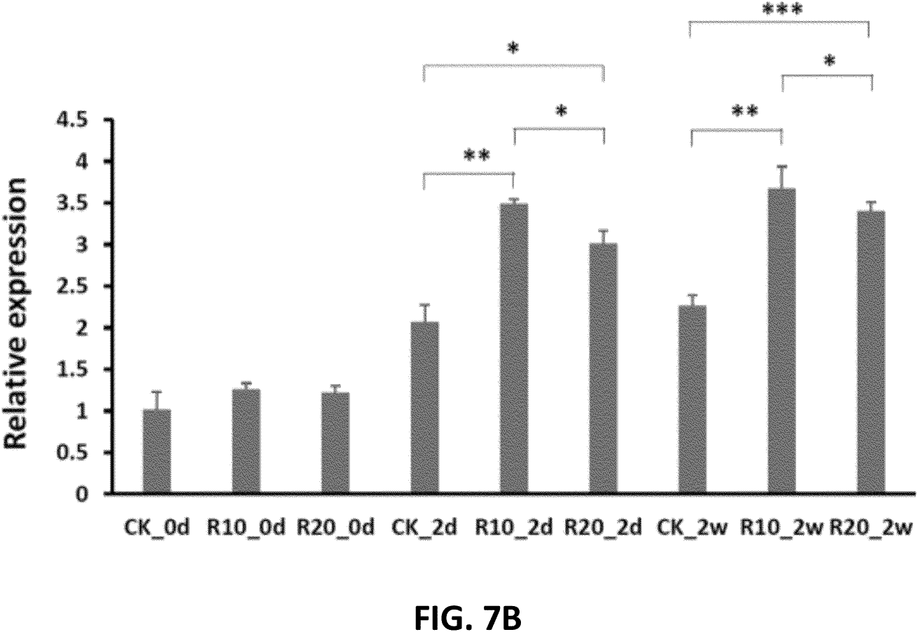

RejuAgro A was found to activate the expression of defense marker genes in citrus. Priming is an active strategy that enhances the defensive response of plants. Priming is also considered as a protective management against Huanglongbing (HLB) infection. To investigate whether RejuAgro A can promote priming activity in citrus, we performed a foliar spray of RejuAgro A on citrus at 0, 10 ppm, and 20 ppm concentrations. Midribs of leaves were collected at 0-day (0d), 2-day (2d), and 2-week (2w). We observed that RejuAgro A triggered the expressions of pathogenesis-related protein CsPR1 (FIG. 7A) and CsPR2 (FIG. 7B). Thus, RejuAgro A promotes priming activity in citrus to combat against HLB.

### Example 7 Media Culture Compositions Used in the Examples

Table 2 includes exemplary media compositions used in the Examples.

TABLE 2

Media compositions

| | | | | |
|---|---|---|---|---|
| Medium Name | Composition | g per liter | pH at 25° C. | Reference |
| YME | Yeast extract | 4.0 g | NA | Hamamoto et. al. (2015) |
| | Malt extract | 10 g | | |
| | Glucose | 4.0 g | | |
| | Tap water | 1.0 L | | |
| | Water | 550 mL | 6.9 | |
| BM7 | Alpha-ketogluterate | 2.0 g | | Fagen et al. (2014) |
| | ACES buffer | 10.0 g | | |
| | KOH | 3.75 g | | |
| | fetal bovine serum (Hyclone) | 150 mL | | |
| | TMN-FH (Hyclone) | 300 mL | | |

### Example 8 Bacterial Strains, Natural Products, and References Cited to Same

The bacterial strains and natural products described in this application and presented in the appended claims are well-known in the microbiology literature. These references are presented below in Table 3 for each of the cited bacterial strains and natural products disclosed herein, the contents of which are hereby incorporated by reference in their entirety.

TABLE 3

Bacterial strains, natural products and references cited in support as evidence of their availability

| | |
|---|---|
| Bacterial Strains | Reference citation |
| 0617-T307, 0917-T305, | Pascual et al. (2014) |
| 0917-T306, and | |
| 0917-T307 | |
| 0118-T319, 0318-T327, | Dabboussi et al. (2002) |
| and 0418-T328 | |
| Bacterial Strains | Reference citation |
| Natural Products | Reference citation |
| RejuAgro A | Present disclosure |

### Citations

Alsohim, A.S., Taylor, T.B., Barrett, G.A., Gallie J., Zhang, X.X., Altamirano-Junqueira, A.E., Johnson, L.J., Rainey, P.B., Jackson, R.W. (2014) The biosurfactant viscosin produced by Pseudomonas fluorescens SBW25 aids spreading motility and plant growth promotion. Environ Microbiol 16:2267-81.

Bové, J. M. (2006). Huanglongbing: A destructive, newly-emerging, century-old disease of citrus. J Plant Pathol, 88(1), 7-37.

Dabboussi, F., Hamze, M., Singer, E., Geoffroy, V., Meyer, J., Izard, D. (2002). Pseudomonas mosselii sp . nov ., a novel species. Int J Syst Bacteriol, 52: 363-376.

Fagen, J. R., Leonard, M. T., McCullough, C. M., Edirisinghe, J. N., Henry, C. S., Davis, M. J., Eric, W. T. (2014). Comparative genomics of cultured and uncultured strains suggests genes essential for free-living growth of Liberibacter. PLoS ONE 9:e84469.

Gwinn, K.D. (2018) Chapter 7 - Bioactive natural products in plant disease control, in: R. Atta ur (Ed.), Studies in Natural Products Chemistry, Elsevier. pp. 229-246.

Hamamoto, H., Urai, M., Ishii, K., Yasukawa, J., Paudel, A., Murai, M., Kaji, T., Kuranaga, T., Hamase, K., Katsu, T., Su, J., Adachi, T., Uchida, R., Tomoda, H., Yamada, M., Souma, M., Kurihara, H., Inoue, M., Sekimizu, K. (2015). Lysocin e is a new antibiotic that targets menaquinone in the bacterial membrane. Nat Chem Biol 11:127-133.

Hu, J., Wright, G. (2019). Huanglongbing of Citrus. Cooperative Extension, The University of Arizona. az1795

Huang, C.Y., Araujo, K., Sanchez, J.N., Kund, G., Trumble, J., Roper, C., Godfrey, K.E., Jin, H. (2021) “A stable antimicrobial peptide with dual functions of treating and preventing citrus Huanglongbing.” Proc Natl Acad Sci USA vol. 118,6: e2019628118.

Masschelein, J., Jenner, M., Challis, G.L. (2017) Antibiotics from Gram-negative bacteria: a comprehensive overview and selected biosynthetic highlights. Nat Prod Rep 34:712-783.

Ma, W., Pang, Z., Huang, X., Xu, J., Pandey, S.S., Li, J., Achor, D.S., Vasconcelos, F.N.C., Hendrich, C., Huang, Y., Wang, W., Lee, D., Stanton, D., Wang, N. (2022). Citrus Huanglongbing is a pathogen-triggered immune disease that can be mitigated with antioxidants and gibberellin. Nat Commun 13, 529.

Pascual, J., García-López, M., Carmona, C., Sousa, T. da S., de Pedro, N., Cautain, B., Martin, J., Vicente, F., Reyes, F., Bills, G. F., & Genilloud, O. (2014). Pseudomonas soli sp. nov., a novel producer of xantholysin congeners. Syst Appl Microbiol, 37: 412-416.

Sena-Vélez, M., Holland, S.D., Aggarwal, M., Cogan, N.G., Jain, M., Gabriel, D.W., Jones, K.M. (2019). Growth Dynamics and Survival of Liberibacter crescens BT-1, an Important Model Organism for the citrus Huanglongbing Pathogen “Candidatus Liberibacter asiaticus.” Appl Environ Microbiol vol. 85: e01656-19.

Tianna, D., Johnson K., Elkins, R., Smith, T., Granatstein, D. (2018). Organic Fire Blight Management in the Western U.S. - eXtension, Organic agriculture.

### Incorporation by Reference

All literature, publications, patents, patent applications, and related material cited here are incorporated by reference as if fully set forth herein.

What is claimed is:

1. A method of controlling a crop disease, comprising the steps of
(i) producing an agricultural composition comprising a Pseudomonas bacterial metabolite as Formula (I) 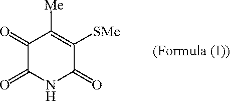 [SMILES: CSC1=C(C)C(=O)C(=O)NC1=O]
; and
(ii) applying the agricultural composition to a crop to inhibit the growth of a pathogenic microorganism.

2. The method of claim 1, wherein the crop disease is selected from the group consisting of citrus Huanglongbing, Zebra chip (ZC) disease of potato, tomato and other plants of the family Solanaceae and plants of the family Apiaceae and Umbelliferae.

3. The method of claim 1, wherein the pathogenic microorganism is selected from the group consisting of Liberibacter crescens, Candidatus Liberibacter asiaticus, Ca. Liberibacter americanus, Ca. Liberibacter africanus, and Ca. Liberibacter solanacearum.

4. The method according to claim 1, wherein the crop is selected from the group consisting of all citrus species and hybrids, as well as numerous citrus relatives in the Family Rutaceae, Solanaceae and other plants of the family Solanaceae, potato, tomato, and plants of the family Apiaceae and Umbelliferae.

5. A method of controlling a crop disease, comprising: applying an agricultural composition comprising between about 1.0 × 10⁵ and 1.0 × 10⁹ cfu per mL Pseudomonas bacteria to a crop to inhibit the growth of a pathogenic microorganism.

6. The method of claim 5, wherein the Pseudomonas bacteria is selected from the group consisting of Pseudomonas soli 0617-T307 (Accession No. PTA-126796), Pseudomonas soli 0917-T305 (Accession No. PTA-126797), Pseudomonas soli 0917-T306 (Accession No. PTA-126798), Pseudomonas soli 0917-T307 (Accession No. PTA-126799), Pseudomonas mosselii 0118-T319 (Accession No. PTA-126800), Pseudomonas mosselii 0318-T327 (Accession No. PTA-126801), and Pseudomonas mosselii 0418-T328 (Accession No. PTA-126802).

7. The method according to claim 5, wherein the agricultural composition comprises between about 5.0 × 10⁷ and 2.0 × 10⁸ cfu per mL Pseudomonas bacteria.

8. The method according to claim 5, wherein the crop disease is selected from the group consisting of citrus Huanglongbing, Zebra chip (ZC) disease of potato, tomato, and other plants of the family Solanaceae and a plant of the family Apiaceae and Umbelliferae.

9. The method according to claim 5, wherein the pathogenic microorganism is selected from the group consisting of Liberibacter crescens, Candidatus Liberibacter asiaticus, Ca. Liberibacter americanus, Ca. Liberibacter africanus, and Ca. Liberibacter solanacearum.

10. The method according to claim 5, wherein the crop is selected from the group consisting of a citrus species or a hybrid derived from the same, a citrus relative in the Family Rutaceae, Solanaceae and a plant of the family Solanaceae, potato, tomato, and a plant of the family Apiaceae and Umbelliferae.

11. A method to activate the expression of a defense marker gene in a citrus crop, comprising the steps of
(i) producing an agricultural composition comprising a Pseudomonas bacterial metabolite as Formula (I) 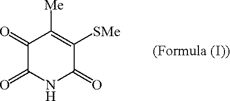 [SMILES: CSC1=C(C)C(=O)C(=O)NC1=O]
; and
(ii) applying the agricultural composition to the citrus crop to activate expression of the defense marker gene.

12. The method of claim 11, wherein the citrus crop is selected from the group consisting of a citrus species, a hybrid derived from a citrus species, a citrus plant species relative in the family Rutaceae, and a citrus plant species relative of the family Solanaceae.

13. The method of claim 11, wherein the defense marker gene is selected from CsPR1 and CsPR2.

14. A method to activate the expression of a defense marker gene in a citrus crop, comprising: applying an agricultural composition comprising between about 1.0 × 10⁵ and 1.0 x 10⁹ cfu per mL Pseudomonas bacteria to the citrus crop to activate the expression of the defense marker gene in the citrus crop.

15. The method of claim 14, wherein the Pseudomonas bacteria is selected from the group consisting of Pseudomonas soli 0617-T307 (Accession No. PTA-126796), Pseudomonas soli 0917-T305 (Accession No. PTA-126797), Pseudomonas soli 0917-T306 (Accession No. PTA-126798), Pseudomonas soli 0917-T307 (Accession No. PTA-126799), Pseudomonas mosselii 0118-T319 (Accession No. PTA-126800), Pseudomonas mosselii 0318-T327 (Accession No. PTA-126801), and Pseudomonas mosselii 0418-T328 (Accession No. PTA-126802).

16. The method according to claim 14, wherein the agricultural composition comprises between about 5.0 × 10⁷ and 2.0 × 10⁸ cfu per mL Pseudomonas bacteria.

17. The method according to claim 14, wherein the citrus crop is selected from the group consisting of a citrus species, a hybrid derived from a citrus species, a citrus plant species relative in the family Rutaceae, and a citrus plant species relative of the family Solanaceae.

18. The method of claim 14, wherein the defense marker gene is selected from CsPR1 and CsPR2.
